SEQUENCE LISTING (10 sequences)

SEQ ID NO: 1 - protein, 5 aa, Homo sapiens
SYAIS

SEQ ID NO: 2 - protein, 17 aa, Homo sapiens
GIGPFFGTANYAQKFQG

SEQ ID NO: 3 - protein, 7 aa, Homo sapiens
DTPYFDY

SEQ ID NO: 4 - protein, 11 aa, Homo sapiens
SGDSIPNYYVY

SEQ ID NO: 5 - protein, 7 aa, Homo sapiens
DDSNRPS

SEQ ID NO: 6 - protein, 11 aa, Homo sapiens
QSFDSSLNAEV

SEQ ID NO: 7 - protein, 116 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSS

SEQ ID NO: 8 - protein, 108 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVL

SEQ ID NO: 9 - protein, 446 aa, Homo sapiens
EVQLVQSGAEVKKPGSSVKVSCKASGGTFSSYAISWVRQAPGQGLEWMGGIGPFFGTANYAQKFQGRVTITADESTSTAYMELSSLRSEDTAVYYCARDTPYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, Homo sapiens
SYELTQPLSVSVALGQTARITCSGDSIPNYYVYWYQQKPGQAPVLVIYDDSNRPSGIPERFSGSNSGNTATLTISRAQAGDEADYYCQSFDSSLNAEVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

(19) United States
(12) Patent Application Publication
(10) Pub. No.: US 2019/0225679 A1
(43) Pub. Date: Jul. 25, 2019

(54) TESIDOLUMAB FOR USE IN THE TREATMENT OF TRANSPLANT REJECTION

(71) Applicant: Novartis AG, Basel (CH)

(72) Inventors: Florian Muellershausen, Basel (CH); Matthias Meier, Grenzach-Wyhlen (DE); Alan Slade, Basking Ridge, NJ (US); Irina Baltcheva, Oberwil (CH); Mark Milton, Belmont (CH)

(21) Appl. No.: 16/306,925

(22) PCT Filed: Jun. 5, 2017
(86) PCT No.: PCT/IB2017/053304

§ 371 Date: Dec. 4, 2018

Related U.S. Application Data
(60) Provisional application No. 62/346,690, filed on Jun. 7, 2016.

(51) Int. Cl. C07K 16/18; A61P 37/06

(52) U.S. Cl. CPC C07K 16/18; A61K 2039/505; C07K 2317/76; A61P 37/06

(57) ABSTRACT

The present invention relates to the use of tesidolumab for the prevention or treatment of transplant rejection and in particular antibody mediated rejection of allografts.

### FIELD OF THE INVENTION

The present invention relates to tesidolumab or an antigen binding fragment thereof for use in the prevention or treatment of transplant rejection or a condition associated thereof such as antibody mediated rejection (AMR), in particular in pre-sensitized patients, as well as adequate weight-based adjusted doses and dosing regimens.

### BACKGROUND OF THE INVENTION

Each year patients are prohibited from receiving a potentially life-saving organ transplant because of pre-existing antibodies directed against the donor's cell surface human leukocyte antigens (HLA). Such patients are considered “sensitized” or “pre-sensitized” to their donor organ, which sensitization may be the result of previous transplantations, pregnancy and/or blood transfusions. The presence of certain donor-specific antibodies (DSA) is a limitation to transplantation regardless of other factors that may indicate a donor match. Within the US and Europe approximately 20-40% of kidney transplant candidates possess donor-specific antibodies (DSA) against Human Leukocyte Antigens (HLA) from potential donor allografts (Siisal and Morath, 2011, Matas et al., 2015). Despite the use of contemporary screening methods, immunosuppressive treatment regimens, priority organ allocation and paired donation programs, pre-sensitized kidney transplant recipients (KTR) rarely receive a renal allograft against which they do not have DSA. For patients with anti-HLA DSA to a donor allograft, the risk of transplant rejection in pre-sensitized KTR remains high and is a significant hurdle to transplantation in this population.

One solution for many pre-sensitized patients is to undergo an HLA-incompatible kidney transplant following antibody depletion by means of desensitization therapies. Transplantation center-specific desensitization protocols include antibody removal by plasmapheresis (PP) or immunoadsorption (IA), antibody modulation through the use of intravenous immunoglobulin (IVIG) and/or occasional off-label use of other immunomodulatory therapy such as B-cell depletion with rituximab or plasma-cell depletion with the proteasome inhibitor bortezomib. These therapeutic strategies have been shown to reduce DSA concentrations sufficiently to facilitate incompatible kidney transplantation. However, the waiting time for a first renal transplant or re-transplantation mainly for highly sensitized candidates for whom a compatible donor allograft cannot be identified remains protracted or even indefinite. For those patients who remain on the transplant waiting list, the continued use of renal replacement therapy (RRT), i.e. maintenance hemodialysis, is associated with increased morbidity and overall mortality, including accelerated progression of cardiovascular disease, higher risk of malignancies as well as decreased quality of life compared to transplanted patients (Montgomery et al., 2011). Kidney transplantation following desensitization conferred a mortality benefit as compared to remaining on maintenance dialysis, such that, the patient survival is 90.6% at 1 year, 85.7% at 3 years, 80.6% at 5 years and 80.6% at 8 years for desensitized KTR compared with the unacceptable rates for wait-listed patients on maintenance dialysis alone of 91.1%, 67.2%, 51.5% and 30.5%, respectively (Montgomery et al., supra). Despite the significant survival benefit realized through HLA-incompatible kidney transplantation of pre-sensitized candidates, post-transplant antibody mediated rejection (AMR) caused by anti-HLA antibodies remains a significant burden that carries a 5.79-fold higher risk of graft loss (95% CI: 3.62-9.24; p<0.001) when compared to HLA compatible matched controls (Orandi et al., (2015) American Journal of Transplantation, 15: 489-498).

AMR is associated with poor long term allograft function and shorter graft survival (Gloor et al., 2010). In a pre-sensitized candidate who receives an HLA-incompatible allograft, complement fixation and activation by DSA bound to allograft endothelium is a key mechanism of AMR, leading to acute and chronic inflammation, vascular damage and graft dysfunction. In the context of kidney transplantation, complement activation is a well-recognized effector mechanism underlying alloantibody-mediated organ rejection and graft loss.

There is no standard, approved, therapy for the prevention or treatment of AMR. Multiple experimental therapeutic approaches have evolved out of necessity and vary from center to center. These approaches may include the administration of corticosteroids, intravenous immune globulin (IVIG), plasmapheresis, immunoadsorption, anti-lymphocyte therapy and altered maintenance immunosuppression or some combination of any of these modalities.

The complement system is a principle component of the innate immune system and represents an important host defense. The complement system and its components enhance the ability of antibodies and phagocytic cells to clear pathogens from an organism, thereby protecting against infection by linking adaptive and innate immunity as well as disposing of immune complexes and the products of inflammatory injury. While important for host defense, dysregulation of complement activity may also cause, or at least contribute to, various diseases. The binding of large amounts of prior to transplantation preformed DSA or post-transplant de novo DSA (dnDSA) to antigens on the endothelial cells of the allograft has been shown to play an important role in acute, subclinical and chronic AMR (Orandi et al., supra). The pathomechanism of acute AMR in pre-sensitized patients is thought to be caused by DSA mediated complement activation on the allograft vascular endothelium, whereas the extent of complement-mediated injury in chronic AMR, however, remains elusive. The three key associations of complement activation in the pathogenesis of AMR include (i) membrane attack complex (MAC) formation via classical pathway activation, leading to direct cell lysis and subsequent vascular damage, inflammation and graft dysfunction; (ii) acute graft injury via the release of chemoattractants (C3a and C5a) and recruitment and activation of inflammatory cells (e.g., neutrophils and macrophages); (iii) direct activation of endothelial cells via C3a and C5a mediated expression of adhesion molecules, cytokines, and chemokines (Colvin and Smith 2005).

In kidney transplantation, C5 blockade through the administration of the anti-C5 antibody eculizumab (Soliris®) has been investigated as a strategy for the prevention of or as a treatment for refractory AMR (Johnson C K, Leca N. (2015) Curr Opin Organ Transplant. 20(6): 643-51). In 2011, Stegall and colleagues reported the first controlled study with short-term eculizumab treatment (12 weeks) in the prevention of acute clinical AMR (Stegall et al, (2011) American Journal of Transplantation 11: 2405-2413). In this trial, 26 pre-sensitized T-and B-cell crossmatch-positive live donor KTR were enrolled and administered eculizumab therapy in addition to standard immunosuppression and induction therapy with rATG. Outcomes in the first 12 months included a significant reduction in acute AMR rates (7%; n=2), as compared to historical controls (44%; n=22/48; P <0.01). In 2015, Cornell and colleagues reported results of an extension to the original trial including longer-term outcomes (>2 years), the treatment of 4 additional KTR (n=30) and administration of 12 months of eculizumab therapy (n=8/30, DSA>200) (Cornell et al, (2015) American Journal of Transplantation 15: 1293-1302). Despite eculizumab treatment, the most frequent histologic abnormality prior to graft loss (n=5/30) was transplant glomerulopathy (TG). While none of the patients who lost their allograft presented with clinical AMR, they all demonstrated anti-HLA Class II DSA with peritubular capillaritis and advanced TG in prior biopsies, 3 of whom received 12 months of eculizumab therapy. Notably, the most important observation from this trial is that, in the setting of persistently high DSA concentrations, such as those in KTR who received long-term eculizumab treatment, eculizumab failed to prevent the development of subclinical inflammation and chronic, microcirculatory injury. However, it is also evident that outcomes were favorable if post-transplant antibody levels were low (Johnson et al., (2015) Curr Opin Organ Transplant. 20(6): 643-51). Due to the inconclusive results of this trial, eculizumab was not developed further for the treatment of AMR.

Furthermore, eculizumab is contraindicated in patients with unresolved serious Neisseria meningitidis infection or in patients who have not been vaccinated against N. meningitidis. Long term administration of eculizumab may be problematic, especially in patients who are particularly sensitive to such infections, e.g. pediatric patients or patients who cannot be vaccinated and therefore, long term administration of eculizumab in these patient groups could increase the risk of infection from N. meningitidis. Transplant patients usually take immunosuppressive treatments for their lifetime and are therefore susceptible to and at risk of contracting opportunist infections. Treatment of these infections in transplant patients is also difficult and more complicated than in non-transplanted patients. Therefore, there remains a need for a safe and effective therapy for preventing or treating AMR, which would improve overall transplant survival for patients receiving cross-match positive transplants. In particular such a therapy would be effective for highly sensitized patients currently deemed unsuitable for transplantation.

The provision of such a therapy for preventing or treating AMR will enable transplantation and improve long term outcomes in presensitized kidney transplant recipients (i.e. it will extend graft function and survival).

### SUMMARY OF THE INVENTION

It is an object of the present invention to provide a medicament for prolonging survival of an allograft. In one aspect, the present invention provides a medicament for the prevention or treatment of transplant rejection or an associated condition such as antibody-mediated rejection (AMR), particularly acute AMR, subclinical AMR and/or chronic AMR or transplant glomerulopathy (TG).

In accordance with the present invention, it has been found that the anti-C5 antibody tesidolumab or an antigen binding fragment thereof is effective in the prevention or treatment of transplant rejection, in particular in the prevention or treatment of acute AMR, subclinical AMR, chronic AMR and/or TG.

Furthermore, adequate weight-based adjusted doses and dosing regimens of tesidolumab (or an antigen binding fragment thereof) have been identified.

Various (enumerated) embodiments of the disclosure are described herein. It will be recognized that features specified in each embodiment may be combined with other specified features to provide further embodiments of the present disclosure.

### Embodiment 1

Tesidolumab or an antigen binding fragment thereof for use in the prevention or treatment of transplantation rejection e.g. in pre-sensitized patients.

### Embodiment 2

Tesidolumab or an antigen binding fragment thereof for use in the prevention or treatment of AMR, e.g. acute AMR, e.g. chronic AMR, or a condition associated thereof.

### Embodiment 3

Tesidolumab or an antigen binding fragment thereof for use in the prevention or treatment of transplant glomerulopathy (TG).

### Embodiment 4

A method of preventing graft rejection and/or prolonging graft survival in a patient in need thereof, e.g. a pre-sensitized patient, comprising administering to said patient a therapeutically effective amount of tesidolumab or an antigen binding fragment thereof.

### Embodiment 5

A method of preventing or treating AMR, e.g. acute AMR, e.g. subclinical AMR, e.g. chronic AMR, or a condition associated thereof, e.g. TG, in a patient in need thereof, comprising administering to said patient a therapeutically effective amount of tesidolumab or an antigen binding fragment thereof.

### Embodiment 6

Tesidolumab or an antigen binding fragment thereof for prolonging survival of an allograft or for the prevention or treatment of a condition associated with transplant rejection such as AMR for patients who are Complement Dependent Cytotoxicity cross-Match (CDC-xM) negative.

### Embodiment 7

Tesidolumab or an antigen binding fragment thereof for prolonging survival of an allograft or for the prevention or treatment of a condition associated with transplant rejection such as AMR for patients characterized by anti-HLA antibody Median Fluorescence Intensity (MFI) (as determined at the day of transplantation) equal to or greater than 5000 or comprised between 2000 and 10000, e.g. between 4000 and 10000 e.g. between 2000 and 8000, e.g. between 3000 and 8000, e.g. between 3000 and 6000, e.g. between 3000 and 5000. Optionally the patient is CDC-xM negative.

### Embodiment 8

Tesidolumab or an antigen binding fragment thereof for prolonging survival of an allograft or for the prevention or treatment of a condition associated with transplant rejection such as AMR for patients characterized by a B-cell flow cytometry cross-match channel shift (BFXM) equal to or greater than 250 or comprised between 150 and 500, Optionally the patient is CDC-xM negative.

### Embodiment 9

Tesidolumab or an antigen binding fragment thereof for prolonging survival of an allograft or for the prevention or treatment of a condition associated with transplant rejection such as AMR for patients characterized by MFI comprised between 2000 and 10000 and BFXM comprised between 150 and 500; or patients characterized by MFI comprised between 4000 and 10000 and BFXM comprised between 150 and 500.

### Embodiment 10

Tesidolumab or an antigen binding fragment thereof for prolonging survival of an allograft or for the prevention or treatment of a condition associated with transplant rejection such as AMR for patients characterized by either MFI equal to or greater than 5000 and BFXM comprised between 150 and 500. Optionally the patient is CDC-xM negative.

### Embodiment 11

Tesidolumab or an antigen binding fragment thereof for prolonging survival of an allograft or for the prevention or treatment of a condition associated with transplant rejection such as AMR for patients characterized by MFI equal to or greater than 5000 and/or (e.g. and) BFXM equal to or greater than 250. Optionally the patient is CDC-xM negative.

### Embodiment 12

Tesidolumab or an antigen binding fragment thereof for prolonging survival of an allograft or for the prevention or treatment of a condition associated with transplant rejection such as AMR for patients characterized by MFI comprised between 2000 and 6000, e.g. comprised between 2500 and 5500, e.g. equal to or greater than 3000 and inferior to 5000. Optionally the patient is CDC-xM negative.

### Embodiment 13

Tesidolumab or an antigen binding fragment thereof for prolonging survival of an allograft or for the prevention or treatment of a condition associated with transplant rejection such as AMR for patients characterized by BFXM equal to or less than 250, e.g. comprised between 150 and 250. Optionally the patient is CDC-xM negative.

### Embodiment 14

Tesidolumab or an antigen binding fragment thereof for prolonging survival of an allograft or for the prevention or treatment of a condition associated with transplant rejection such as AMR for patients characterized by MFI comprised between 3000 and 5000 and BFXM less than 250. Optionally the patient is CDC-xM negative.

### Embodiment 15

Tesidolumab or an antigen binding fragment thereof for prolonging survival of an allograft or for the prevention or treatment of a condition associated with transplant rejection such as AMR for patients characterized by MFI equal to or greater than 3000 and less than 5000 and BFXM is equal to or greater than150 and less than 250. Optionally the patient is CDC-xM negative.

### Embodiment 16

A dosing regimen of tesidolumab, or an antigen binding fragment thereof, for prolonging survival of an allograft or for the prevention and/or treatment of a condition associated with transplant rejection such as AMR, wherein tesidolumab or said antigen binding fragment thereof is (e.g. is to be) administered at a dose of at least 20 mg/kg weekly for a period of at least one month, e.g. at least 3 months, e.g. at least 6 months, e.g. at least one year, e.g. lifelong. In another embodiment, tesidolumab or said antigen binding fragment thereof is (e.g. is to be) administered at a dose of at least 20 mg/kg weekly for the first week or the first two weeks of treatment.

### Embodiment 17

A dosing regimen of tesidolumab or an antigen binding fragment thereof, for prolonging survival of an allograft or for the prevention and/or treatment of a condition associated with transplant rejection such as AMR, wherein tesidolumab or said antigen binding fragment thereof is (e.g. is to be) administered at a dose of at least 20 mg/kg every two weeks for a period of at least one month, e.g. at least 3 months, e.g. at least 6 months, e.g. at least one year, e.g. lifelong.

### Embodiment 18

Tesidolumab or an antigen binding fragment thereof, for prolonging survival of an allograft or for the prevention or treatment of a condition associated with transplant rejection such as AMR, wherein tesidolumab or said antigen binding fragment thereof is (e.g. is to be) administered repeatedly at a dose of at least 20 mg/kg and wherein the interval between two administrations is less than one month, e.g. is 2 weeks.

### Embodiment 19

Tesidolumab or an antigen binding fragment thereof, for prolonging survival of an allograft or for the prevention or treatment of a condition associated with transplant rejection such as AMR, wherein tesidolumab or said antigen binding fragment thereof is (e.g. is to be) administered at a dose of at least 20 mg/kg weekly for a period of at least 2 weeks to 6 months, and is then administered at a dose of at least 20 mg/kg every two weeks for at least 3 months.

### Embodiment 20

Tesidolumab or an antigen binding fragment thereof, for prolonging survival of an allograft or for the prevention or treatment of a condition associated with transplant rejection such as AMR, wherein tesidolumab or said antigen binding fragment thereof is (e.g. is to be) administered as at least one (e.g. one) induction dose of least about 40 mg/kg prior to transplantation, e.g. up to 12 hours prior to transplantation, e.g. up to 10 hours, e.g. up to 8 hours, e.g. up to 6 hours prior to transplantation, or at the time of transplantation.

### Embodiment 21

Tesidolumab or an antigen binding fragment thereof for use in the prevention or treatment of AMR or an associated condition thereof (e.g. TG) in a patient, wherein tesidolumab or said antigen binding fragment thereof is administered such that a constant plasma trough level at steady-state of total antibody of 10-100 μg/mL is maintained, e.g. 50-100 μg/mL, e.g. 55-100 μg/mL, e.g. 50-60 μg/mL. In a specific embodiment the condition is acute AMR. In another embodiment the condition is chronic AMR or TG.

### Embodiment 22

Tesidolumab or an antigen binding fragment thereof, for prolonging survival of an allograft or for use in the prevention or treatment of a condition associated with transplant rejection such as AMR, wherein tesidolumab or an antigen binding fragment thereof is (e.g. is to be) administered at a dose of at least 20 mg/kg weekly for a period of at least 2 weeks to 6 months, and then is administered at a dose of at least 20 mg/kg every two weeks for at least 3 months, 6 months, 9 months, 1 year, lifelong.

### Embodiment 23

Tesidolumab or antigen binding fragment thereof for prolonging survival of an allograft or for the use in the prevention or treatment of AMR or an associated condition thereof, wherein said antibody is administered at a dose of at least 20 mg/kg and wherein the interval between two consecutive administrations is comprised between 1 week and one month, e.g. is of 1 week, and then the interval between two consecutive administrations is increased, e.g. two times, during the second period of treatment.

### Embodiment 24

Tesidolumab or an antigen binding fragment thereof for use prolonging survival of an allograft or in the prevention or treatment of AMR, wherein the patient has MFI comprised between 2000 and 10000 and/or BFXM is between 150 and 500, e.g. MFI greater than 5000 and/or (e.g. and) BFXM greater or equal than 250, wherein tesidolumab or said antigen binding fragment thereof is administered at a dose of at least 20 mg/kg weekly for a period of at least 1 week, followed by at least 20 mg/kg every two weeks for a period of at least 6 weeks. The total treatment duration can be of at least 6 months, or one year.

### Embodiment 25

Tesidolumab or an antigen binding fragment thereof for use in prolonging survival of an allograft or in the prevention or treatment of AMR, wherein the patient has MFI of 3000 to 5000 and/or (e.g. and) BFXM less than 250, wherein tesidolumab or said antigen binding fragment thereof is administered at a dose of at least 20 mg/kg weekly for a period of at least 1 week, followed by at least 20 mg/kg every two weeks for a period of at least 6 weeks. The total treatment duration can be of at least 6 months or one year.

### Embodiment 26

Tesidolumab or an antigen binding fragment thereof for use prolonging survival of an allograft or in the prevention or treatment of AMR wherein the patient is a solid organ transplant patient, e.g. a kidney transplant patient. In particular the patient is characterized by MFI of 3000 to 5000 and/or (e.g. and) BFXM equal to or less than 250. Or the patient is characterized by MFI greater than 5000 and/or (e.g. and) BFXM equal to or greater than 250.

### Embodiment 27

Tesidolumab or antigen binding fragment thereof for prolonging survival of an allograft or for the use in the prevention or treatment of AMR or an associated condition thereof in a patient, wherein said antibody is administered at a dose of at least 20 mg/kg and wherein the interval between two consecutive administrations is comprised between 1 week and one month, e.g. is of 1 week, and then the interval between two consecutive administrations is increased, e.g. two times, during the second period of treatment, and wherein said patient is characterized by MFI comprised between 3000 and 5000 and/or (e.g. and) BFXM equal to or less than 250.

### Embodiment 28

Tesidolumab or antigen binding fragment thereof for prolonging survival of an allograft or for the use in the prevention or treatment of AMR or an associated condition thereof in a patient, wherein said antibody is administered at a dose of at least 20 mg/kg and wherein the interval between two consecutive administrations is comprised between 1 week and one month, e.g. is of 1 week, and then the interval between two consecutive administrations is increased, e.g. two times, during the second period of treatment, and wherein said patient is characterized by MFI greater than 5000 and/or (e.g. and) BFXM equal to or greater than 250.

### Embodiment 29

Use of tesidolumab or an antigen binding fragment thereof for the manufacture of a medicament (a) for prevention of transplant rejection e.g. in pre-sensitized patients, or (b) for the prevention or treatment of AMR, e.g. acute AMR, e.g. chronic AMR, or a condition associated thereof, e.g. transplant glomerulopathy (TG). In particular the patient is characterized by MFI of 3000 to 5000 and/or (e.g. and) BFXM less than 250. Or the patient is characterized by MFI greater than 5000 and/or (e.g. and) BFXM greater or equal than 250,

### Embodiment 30

A method of preventing a transplantation rejection, or of preventing or treating AMR, e.g. acute AMR, e.g. chronic AMR, or a condition associated thereof, e.g. TG, in a patient in need thereof, comprising administering to said patient weight-based adjusted doses of tesidolumab or an antigen binding fragment thereof to said patient of at least 20 mg/kg. In particular the patient is characterized by MFI (as determined on the day of transplantation) of 3000 to 5000 and/or (e.g. and) BFXM less than 250. Or the patient is characterized by MFI greater than 5000 and/or (e.g. and) BFXM greater or equal than 250.

### DETAILED DESCRIPTION OF THE INVENTION

For the condition of AMR, the cellular and molecular pathways are under investigation; however current knowledge of humoral immunobiology indicates that B cell and plasma cell activation results in the generation of DSA, which bind to HLA or non-HLA molecules on the endothelium. Antibody binding to endothelium and subsequent cellular activation involving complement-dependent and -independent pathways leads to the recruitment of natural killer (NK) cells, polymorphonuclear neutrophils and macrophages, which contribute to capillaritis and eventual tissue injury (Farkash & Colvin (2012) Nat Rev Nephrol., 8:255-7; Sis & Halloran (2010) Curr Opin Organ Transplant., 15: 42-8; Hidalgo et al (2010) Am J Transplant., 10: 1812-22).

AMR is differentiated into acute, subclinical and chronic AMR. The diagnosis requires histologic evidence from a kidney biopsy demonstrating acute or chronic tissue injury, evidence of current/recent antibody interaction with vascular endothelium and serologic evidence of the presence of circulating DSA (Haas et al., 2014). Clinically, the diagnosis of AMR is generally preceded by an acute and/or chronic change in renal function. These functional changes are the basis for obtaining an allograft biopsy that may result in the diagnosis of acute and/or chronic AMR. Although, the Banff criteria (Solez K et al., (1993) Kidney International, 44: 411-22) do not incorporate allograft function in the diagnosis of AMR, the transplant community has adopted additional terminology to further differentiate acute and chronic AMR.

Acute Clinical AMR: Acute clinical episodes of AMR are defined as those that have evidence of graft dysfunction, manifested as oliguria/anuria, an increase in serum creatinine by more than 20% from baseline, the need for hemodialysis more than 7 days post-transplant, or new onset proteinuria at the time of the AMR-defining biopsy (per Banff 2013 classification; Haas et al. (2014) Am J Transplant. 14(2): 272-83).

Subclinical AMR: Subclinical episodes of AMR (scAMR) include all of the histopathologic hallmark features of acute AMR as per the Banff 2013 classification, without the clinical presentation of graft dysfunction, mainly stable serum creatinine

Chronic AMR: Chronic AMR results from a repetitive pattern of chronic thrombotic events and inflammatory changes, which result in cellular injury and repair. It manifests as late transplant glomerulopathy (TG) and results in a decline in renal function. Chronic AMR is measured by histological parameters as per the Banff 2013 classification and defined as the presence (cg>1) or absence (cg=0) of transplant glomerulopathy (TG) on kidney biopsies performed over time following kidney transplantation.

Transplant glomerulopathy (TG, also known as or chronic allograft glomerulopathy) is a disease of the glomeruli in transplanted kidneys. TG is characterized by glomerular mesangial expansion and capillary basement membrane (BM) duplication, seen as basement membrane double contouring or splitting. The prognosis of transplant glomerulopathy is poor. Within 5 years of diagnosis, the death-censored graft survival rate is as low as 20% (John Ret al., (2010) Transplantation 90: 757-764). TG is most often associated with chronic AMR and DSA; however it has also been associated with hepatitis C, chronic thrombotic microangiopathy and autoimmune conditions.

There is a clear need for safe treatments adapted to transplanted patients, in particular pre-sensitized patients, that are able to prolong survival of an allograft and provide efficacious prophylaxis or treatment of conditions associated with transplant rejection, such as antibody-mediated rejection (AMR), particularly acute AMR, subclinical AMR, chronic AMR or transplant glomerulopathy (TG). As discussed previously, eculizumab treatment did not show efficacy in the treatment of AMR.

In the present invention, it was found that tesidolumab or an antigen binding fragment thereof is effective in the prevention or treatment of transplant rejection, AMR or an associated condition, in particular in the prevention or treatment of acute AMR, chronic AMR, e.g. TG.

In one aspect, the present invention relates to the anti-C5 antibody tesidolumab or an antigen binding fragment thereof for use in the prevention and/or treatment of transplant rejection. The patient may be pre-sensitized patients. The present invention also relates to tesidolumab or an antigen binding fragment thereof for use in the prevention or treatment of AMR or an associated condition. In one embodiment, the present invention relates to tesidolumab or an antigen binding fragment thereof for use in the prevention or treatment of acute AMR. In a specific, preferred embodiment, the present invention relates to tesidolumab or an antigen binding fragment thereof for use in the prevention or treatment of chronic AMR or a condition associated thereof, e.g. transplant glomerulopathy (TG).

As defined herewith, “sensitized” or “pre-sensitized” patients refers to patients who have pre-existing donor-specific antibodies (DSA) and in particular antibodies directed against the donor's cell surface human leukocyte antigens (HLA).

An allograft according to the disclosure can include a transplanted organ, part of an organ, tissue or cell. These include, but are not limited to, heart, kidney, lung, pancreas, liver, vascular tissue, eye, cornea, lens, skin, bone marrow, muscle, connective tissue, gastrointestinal tissue, nervous tissue, bone, stem cells, islets, cartilage, hepatocytes, and hematopoietic cells. In one embodiment, the patient is a solid organ transplant patient, preferably a kidney transplant patient. The term “solid organ”, as used herein, refers to an internal organ that has a firm tissue consistency and is neither hollow (such as the organs of the gastrointestinal tract) nor liquid (such as blood). Such organs include the heart, kidney, liver, lungs, and pancreas.

Tesidolumab is a recombinant, high-affinity, human monoclonal antibody of the IgG1/lambda isotype, which binds to C5 and neutralizes its activity in the complement cascade. As described previously, C5 serves as a central node necessary for the generation of C5a as well as the formation of the membrane attack complex (MAC, C5b-9).

Tesidolumab is described in Intl. Pat. Appl. No. WO 2010/015608 “Compositions and Methods for Antibodies Targeting Complement Protein C5” and U.S. Pat. No. 8,241,628.

The CDR sequences of tesidolumab are included herein in Table 1: HCDR1 sequence (SEQ ID NO: 1), HCDR2 sequence (SEQ ID NO: 2), HCDR3 sequence (SEQ ID NO: 3), LCDR1 sequence (SEQ ID NO: 4), LCDR2 sequence (SEQ ID NO: 5) and LCDR3 sequence (SEQ ID NO: 6), numbered according to Kabat definition. The VH and VL sequences and full length heavy and light chain sequences are given in Table 1 as SEQ ID Nos: 7-10, respectively.

In another embodiment, the anti-C5 antibody to be administered is any antibody having the CDR sequences of tesidolumab, as described in SEQ ID Nos: 1-6, or a homology of at least 95% thereof.

It is to be noted that eculizumab and tesidolumab, whilst both binding to the human C5 complement protein, have different nucleic and amino acid sequences and furthermore do not bind to the same epitope on the human C5 complement protein. Eculizumab is a humanized antibody, whereas tesidolumab is fully human monoclonal antibody. It has been acknowledged by health authorities that these antibodies are not similar antibodies.

The term “treating” or “treatment” as used herein includes the administration of antibodies to prevent or delay the onset of the symptoms, complications, or biochemical indicia of a disease (e.g. AMR), alleviating the symptoms or arresting or inhibiting further development of the disease, condition, or disorder. Treatment may be prophylactic (to prevent or delay the onset of the disease, or to prevent the manifestation of clinical or subclinical symptoms thereof) or therapeutic suppression or alleviation of symptoms after the manifestation of the disease. Within the meaning of the present invention, the term “treat” also denotes to arrest, delay the onset (i.e., the period prior to clinical manifestation of a disease) and/or reduce the risk of developing or worsening a disease. The term “prevent” or “prevention” refers to a partial or complete inhibition of development or progression of a disease

The terms “individual”, “host”, “subject”, and “patient” are used interchangeably to refer to the human patient that is the object of treatment, observation and/or experiment. According to the invention, the patient is an organ transplant patient, e.g. a solid organ transplant patient, or can be a patient waiting for a transplant, e.g. a transplant candidate, e.g. a solid organ transplant candidate. For example, the patient is a kidney transplant or a kidney transplant candidate.

The patient can be “sensitized” or “pre-sensitized”. The patient can be of high risk or medium risk of AMR, as hereinabove defined. In another embodiment, the patient may already have had a transplant before.

An ever growing gap between the number of patients requiring organ transplantation and the number of donor organs available has become a major problem throughout the world (Park W D et al. (2003) Am. J Transplant 3:952-960). Individuals who have developed anti-HLA antibodies are said to be immunized or sensitized (Gloor (2005) Contrib. Nephrol. 146:

11-21). HLA sensitization is the major barrier to optimal utilization of organs from living donors in clinical transplantation (Warren et al. (2004). Am. J Transplant. 4:561-568) due to the development of severe AMR. For example, more than 50% of all individuals awaiting kidney transplantation are presensitized patients (Glotz D et al., (2002) Am. J. Transplant. 2: 758-760) who have elevated levels of broadly reactive alloantibodies, resulting from multiple transfusions, prior failed allografts, or pregnancy (Kupiec-Weglinski, (1996) Ann. Transplant. 1: 34-40). The role of AMR is currently one of the most dynamic areas of study in transplantation, due to recognition that this type of rejection can lead to either acute and/or chronic loss of allograft function (Mehra et al., (2003) Curr. Opin. Cardiol. 18: 153-158). The quantity (titre) as well as the avidity of circulating DSAs is a major factor influencing the clinical expression of AMR therefore determining the level of sensitization at the time of transplantation is a key inclusion criterion for transplant patients.

Laboratories can use a number of methods for determining the presence of DSAs in a patient. Recent developments have enabled a more accurate prediction of transplantation success utilizing assays that permit recognition of autologous and non-HLA antibodies, more sensitive cross matching techniques, flow cytometry and the use of solid-phase immunoassays (SPI) such as single antibody beads (SAB) assays to identify antibody specificity with greater precision and sensitivity (Kerman R H et al., (1996) Transplantation 62: 201; Lee P A et al., (2007) In: Clinical Transplants. Los Angeles: The Terasaki Foundation Laboratory pp 219). For solid-phase immunoassays it is relevant to capture both the HLA antibody specificities identified and the level of antibody (mean fluorescence index; MFI). Donor-specific antibody (DSA) concentrations can be measured by a Luminex single antigen bead (SAB) assay. MFI levels on the beads represent the amount of antibody bound relative to the total antigen present on the beads (degree of saturation), which varies by individual bead. Immunologic risk assessment can be given by listing antibody specificities according to the MFI ranges of low, medium or high. Flow cytometry is a sensitive technique useful in identifying patients with weak DSA who are at increased risk of AMR and graft rejection (Couzi et al (2011) Transplantation, 91: 527). B-cell flow cytometry cross-match channel shift (BFXM) identifies antibodies binding to target lymphocytes through a method involving a fluorescent secondary antibody and quantification via a flow cytometer.

The use of the two systems in combination, MFI by single antibody bead assays and BFXM, permits the measurement of DSA titres in a sample, even low titres, while determining the avidity of the DSAs. The combination of the BFXM and SAB MFI tests allows for better and more accurate separation between patients based on their AMR risk than would be possible by using each method alone.

High-risk candidates (high-risk sensitization level) are defined as those who are Complement dependent cytotoxicity cross-match (CDC-xM) negative with anti-HLA SAB MFI on the day of transplantation (highest single antigen) equal to or greater than 5000 and a positive mean BFXM channel shift of equal to or greater than 250. Moderate-risk candidates (moderate-risk sensitization level) will be defined as those who are CDC-xM negative with anti-HLA antibody SAB MFI on the day of transplantation (highest single antigen) equal to or greater than 3000 and less than 5000 and a positive mean BFXM channel shift of less than 250.

Complement dependent cytotoxicity (CDC) is a function of the complement system. CDC refers to the lysis of a target cell in the presence of complement system proteins. The presence of positive complement-dependent cytotoxicity crossmatches (CDC-xM) generally has been considered as a contraindication to kidney transplantation.

Historically, the presence of DSAs pre-transplantation was a contraindication for transplantation and as a result many highly sensitized patients did not receive a transplant due to the positive serologic cross match with nearly all donors. With the introduction of more sensitive SPI, the number of highly sensitized patients increased; however the presence of DSAs is no longer seen as a contraindication but rather as a risk factor for graft rejection and loss. As such, the risk can be decreased by either selecting a donor for which the patient has no DSAs or removal of the DSAs by desensitization protocols. One solution for many pre-sensitized patients is to undergo an HLA-incompatible kidney transplant following antibody depletion using desensitization strategies. Transplantation center specific desensitization protocols, include antibody removal by plasmapheresis or immunoadsorption, antibody modulation through the use of intravenous immunoglobulin (IVIG) and/or occasional off-label use of other immunomodulatory therapy such as B-cell depletion with rituximab or plasma-cell depletion with the proteasome inhibitor bortezomib. These therapies have been shown to reduce DSA concentrations sufficiently to facilitate incompatible kidney transplantation (Legendre et al., (2013) Transplant Rev. 27(3): 90-2).

In one embodiment, the present invention relates to tesidolumab or an antigen binding fragment for use in the prevention or treatment of a disease selected from transplantation rejection, AMR, e.g. acute AMR, e.g. chronic AMR, or a condition associated thereof, e.g. transplant glomerulopathy (TG), wherein the patient is characterized by MFI (as determined on the day of transplantation) comprised between 2000 and 10000 and/or BFXM comprised between 150 and 500, e.g. MFI comprised between 3000 and 5000 and/or BFXM less than 250. In a preferred embodiment, the patient is characterized by MFI (as determined on the day of transplantation) comprised between 2000 and 10000 and BFXM comprised between 150 and 500, e.g. MFI comprised between 3000 and 5000 and BFXM less than 250.

In one embodiment, the patient is CDC-crossmatch negative.

In one embodiment, the present invention relates to a solid organ transplant patient, preferably to a kidney transplant patient. In a further embodiment, the present invention relates to a solid organ transplant patient, preferably to a kidney transplant patient, characterized by MFI (as determined on the day of transplantation) comprised between 2000 and 10000 and/or BFXM comprised between 150 and 500, e.g. MFI comprised between 3000 and 5000 and/or BFXM less than 250. In a preferred embodiment, the present invention relates to a solid organ transplant patient, preferably to a kidney transplant patient, characterized by MFI (as determined on the day of transplantation) comprised between 2000 and 10000 and BFXM comprised between 150 and 500, e.g. MFI comprised between 3000 and 5000 and BFXM less than 250.

The term “an effective amount” or “therapeutically effective amount” of an anti-C5 antibody or antigen binding fragment thereof refers to an amount of the anti-C5 antibody or antigen binding fragment of the present disclosure that will elicit a biological or medical response in a patient, for example, reduction or inhibition of a protein activity, or ameliorate symptoms, alleviate conditions, slow or delay disease progression, or prevent a disease, etc. The term “effective amount” or “therapeutically effective amount” is defined herein to refer to an amount sufficient to provide an observable improvement over the baseline clinically observable signs and symptoms of the condition treated.

The term “about” or “approximately” shall have the meaning of within 10%, more preferably within 5%, of a given value or range.

In a method according to the invention, there is provided the administration of a maintenance dose of tesidolumab or an antigen binding fragment thereof, for treating or preventing AMR or a condition associated thereto, e.g. acute AMR, e.g. chronic AMR, e.g. TG.

The maintenance dose is comprised of between 10 mg/kg and 50 mg/kg, e.g. between 10 mg/kg and 40 mg/kg, e.g. between 10 mg/kg and 30 mg/kg, e.g. is about 15 mg/kg, 20 mg/kg, 25 mg/kg, 30 mg/kg, 35 mg/kg.

In certain embodiments, the maintenance dose is administered 1, 2, 3, 4, 5, 6 or more times, or from 1 to 3, 1 to 4, 2 to 4, 2 to 5, 2 to 6, 3 to 6, 4 to 6, 6 to 8, or more times.

In some embodiments, the maintenance dose is administered at least weekly, at least every two weeks, at least monthly.

The period during which the maintenance dose is administered to the patient is herein referred to as the maintenance period. During the maintenance period, the maintenance dose can be supplemented by at least one supplemental dose, as described herein below.

The maintenance period can start prior the transplantation, at the day of the transplantation or after the transplantation, e.g. one week, two weeks or one month after the transplantation.

The duration of administration of the maintenance dose, e.g. duration of the maintenance period, is at least 6 weeks, e.g. at least 9 weeks, e.g. at least 3 months, e.g. at least 6 months, e.g. at least 9 months, e.g. at least one year, e.g. lifelong. The maintenance period can last until the transplant patient need a new transplantation.

In some embodiments, tesidolumab or an antigen binding fragment thereof is administered in such a way that a constant serum trough level of tesidolumab or an antigen binding fragment thereof of at least approximately 10 μg/mL, e.g. at least approximately 20 μg/mL, e.g. at least approximately 30 μg/mL, e.g. at least approximately 40 μg/mL, e.g. at least approximately 50 μg/mL, e.g. at least approximately 55 μg/mL, is achieved.

As herein above defined, serum trough level of tesidolumab or an antigen binding fragment thereof refers to the serum trough level of total antibody (or an antigen binding fragment thereof), free antibody or bond antibody, e.g. to total antibody (i.e. antibody that is free plus antibody that is bound to the serum C5 complement protein).

In some embodiments, tesidolumab or an antigen binding fragment thereof is administered in such a way that a constant serum trough level of tesidolumab or an antigen binding fragment thereof of 10-100 μg/mL is maintained, e.g. 20-100 μg/mL, e.g. 30-100 μg/mL, e.g. 40-100 μg/mL, e.g. 50-100 μg/mL, e.g. 55-100 μg/mL, e.g. 50-60 μg/mL, e.g. about 55 μg/mL.

In other embodiments, tesidolumab or an antigen binding fragment thereof is administered in such a way that a constant serum trough concentration of at least 10 μg/mL, e.g. at least 20 μg/mL, e.g. at least 30 μg/mL, e.g. at least 40 μg/mL, e.g. at least 50 μg/mL, preferably at least 55 μg/mL, more preferably at least 100 μg/mL, e.g. at least 200 μg/mL, is achieved.

In specific embodiments, the dose may be increased if the trough concentration (e.g. in serum) of tesidolumab or an antigen binding fragment thereof (e.g. of total antibody) in the patient is below 10 μg/mL, e.g. below 20 μg/mL, e.g. below 30 μg/mL, e.g. below 40 μg/mL, e.g. below 50 μg/mL, e.g. below 55 μg/mL, e.g. below 60 μg/mL, e.g. below 70 μg/mL, e.g. below 80 μg/mL, e.g. below 90 μg/mL, or e.g. below 100 μg/mL.

In specific embodiments, the dose is decreased if the trough concentration (e.g. in serum) of tesidolumab or an antigen binding fragment thereof (e.g. of total antibody) from the patient is above 50 μg/mL, e.g. above 55 μg/mL, e.g. above 100 μg/mL, e.g. above 150 μg/mL, e.g. above 200 μg/mL, e.g. above 300 μg/mL, e.g. above 400 μg/mL, or e.g. above 500 μg/mL.

In specific embodiments, the dose is maintained if the trough concentration (e.g. in serum) of tesidolumab or an antigen binding fragment thereof (e.g. of total antibody) from the patient is 10-100 μg/mL, e.g. 50-100 μg/mL, e.g. 55 μg/mL to 100 μg/mL.

According to the invention, tesidolumab or antigen binding fragment thereof, is administered to a patient at the maintenance dose at least weekly, or at least every two weeks or at least monthly.

The maintenance dose can be administered over a period of at least 6 weeks, e.g. at least 9 weeks, e.g. at least 3 months, e.g. at least 6 months, e.g. at least 9 months, e.g. at least one year, e.g. lifelong.

In one embodiment, tesidolumab or antigen binding fragment thereof, is administered to a patient during a maintenance period every two weeks (e.g. as an infusion) at a dose of about 20 mg/kg. The period during which the maintenance dose is administered lasts for a period of at least 6 weeks, e.g. 3 months, e.g. 6 months, e.g. 9 months, e.g. one year, e.g. lifelong.

As used herein, the terms “trough level” and “trough concentration” refer to the lowest levels of free anti-C5 antibody or antigen binding fragment thereof in a sample (e.g., a serum or plasma sample, e.g. serum) from a patient over a period of time. In certain embodiments, the period of time is the entire period of time between the administration of one dose of tesidolumab or antigen binding fragment thereof and another dose of said tesidolumab or antigen binding fragment thereof. In some embodiments, the period of time is approximately 24 hours, approximately 48 hours, approximately 72 hours, approximately 7 days, or approximately 14 days after the administration of one dose of tesidolumab or antigen binding fragment thereof and before the administration of another dose of tesidolumab or antigen binding fragment thereof.

According to the invention, there is provided a dose of tesidolumab or antigen binding fragment thereof, such that the concentrations of serum tesidolumab, e.g. constant serum trough level at steady-state of antibody, e.g. constant serum trough level at steady-state of total antibody, is comprised between 10 and 100 μg/mL, e.g. 50 and 100 μg/mL, e.g. 55 to 100 μg/mL, e.g. 40 to 60 μg/mL, e.g. 45 to 55 μg/mL. For example, the concentration of total serum tesidolumab, e.g. constant serum trough level at steady-state of total antibody, is about 100 μg/mL, e.g. about 60 μg/mL, e.g. about 55 μg/mL, e.g. about 50 μg/mL.

According to the present invention, tesidolumab or antigen binding fragment thereof is administered repeatedly.

The term “repeated administration”, as used herein, refers to administration of the anti-C5 antibody of the invention, e.g. tesidolumab, at an administration interval between two administrations of not more than one month, e.g. not more than three weeks, e.g. not more than two weeks, e.g. not more than one week, e.g. for at least 3 months, e.g. for at least 6 months, e.g. for at least 9 months, e.g. for at least 1 year, e.g. for lifelong.

According to the present invention, a first maintenance dose of tesidolumab or an antigen binding fragment thereof is administered to the patient prior to or after transplantation, e.g. at the time of transplantation, e.g. one week after transplantation, e.g. two weeks after transplantation.

In some embodiments, an induction dose of tesidolumab or an antigen binding fragment thereof is administered to the patient, e.g. before or after the transplantation, e.g. at the time of transplantation, e.g. prior to transplantation, e.g. up to 12 hours, e.g. up to 10 hours, e.g. up to 8 hours, e.g. up to 6 hours prior to transplantation.

According to the invention, the induction dose is defined as a dose higher than the maintenance dose. As herein above defined, the induction phase is the period at the beginning of treatment during which the dose of tesidolumab, or an antigen binding fragment thereof, that is administered to the patient, is higher than the maintenance dose. The induction phase is optional. It can last for at least one week, e.g. one week, e.g. two weeks, e.g. one month. It can start before transplantation, at the day of transplantation or after transplantation, e.g. at the day of the transplantation.

The induction dose of tesidolumab or an antigen binding fragment thereof is between 30 mg/kg and 100 mg/kg, e.g. 40-80 mg/kg, e.g. 40 mg/kg, e.g. 50 mg/kg. In certain embodiments, the induction dose is administered 1, 2, 3, 4, 5, 6 or more times, or 1 to 3, 1 to 4, 2 to 4, 2 to 5, 2 to 6, 3 to 6, 4 to 6 or 6 to 8 times. In some embodiments, the induction dose is administered 1, 2, 3, 4, 5, 6 or more times, or 1 to 3, 1 to 4, 2 to 4, 2 to 5, 2 to 6, 3 to 6, 4 to 6 or 6 to 8 times over a 5 to 7 day, 5 to 10 day, 7 to 12 day, 7 to 14 day, 7 to 21 day or 14 to 21 day period of time.

In certain embodiments, the induction dose is 1.2, 1.25, 1.3, 1.35, 1.4, 1.45, 1.5, 2, 2.5, 3, 3.5, 4, 4.5, 5, 5.5, or 6 times higher than the maintenance dose, or 1.2 to 2, 2 to 3, 2 to 4, 2 to 6, 3 to 4, 3 to 6, or 4 to 6 times higher than the maintenance dose.

In some embodiments, the maintenance dose is 25%, 30%, 35%, 40%, 45%, 50%, 55%, 60%, 65%, 70%, 75%, 80%, 85%, 90%, 95%, 100%,105%, 110%, 115%, 120%, 125%, 130%, 135%, 140%, 145%, 150%, 155%, 160%, 165%, 170%, 175%, 180%, 185%, 190%, 195%, or 200% lower than the induction dose.

According to the invention, there is provided a dosing regimen comprising (a) administering at least one induction dose of the anti-C5 antibody of the present invention, e.g. tesidolumab, to a patient; and (b) administering a maintenance dose of said antibody.

In one embodiment, provided herein is a method for prolonging graft survival or for preventing or treating AMR or a condition associated thereof, e.g. acute AMR, e.g chronic AMR, e.g. TG, in a patient in need thereof, the method comprising:
(a) administering at least one induction dose of the anti-C5 antibody of the present invention, e.g. tesidolumab, to a patient, e.g. prior to or on the day of transplantation; and
(b) administering a maintenance dose of said antibody repeatedly, e.g. tesidolumab, to the patient e.g. in such a way that the constant trough concentration of said antibody is 10-100 μg/mL, e.g. 50-100 μg/mL, e.g. 55-100 μg/mL, e.g. 50-60 μg/mL, e.g. about 55 μg/mL.

In one embodiment, the dosing regimen comprises administration of tesidolumab or an antigen binding fragment thereof, to a patient, e.g. a transplant candidate,
(a) at least one induction dose of at least about 30 mg/kg, preferably at least about 40 mg/kg, e.g. about 50 mg/kg, e.g. about 60 mg/kg, e.g. about 70 mg/kg, e.g. about 80 mg/kg, e.g. about 90 mg/kg, e.g. about 100 mg/kg, prior to transplantation, e.g. up to 12 hours, e.g. up to 10 hours, e.g. up to 8 hours, e.g. up to 6 hours prior to transplantation, or at the time of transplantation;
(b) followed by at least two weekly maintenance doses, e.g. three weekly maintenance doses, e.g. 4 weekly maintenance doses, e.g. 5 maintenance weekly doses, e.g. 6 weekly maintenance doses, of at least about 20 mg/kg, e.g. about 25 mg/kg, e.g. about 30 mg/kg, e.g. about 40 mg/kg, of said anti-C5 antibody.

In a preferred embodiment, the dosing regimen comprises administering tesidolumab or an antigen binding fragment thereof, e.g. tesidolumab, to a patient (e.g. a transplant candidate) at least one induction dose of about 40 mg/kg within a time period from up to six hours prior to transplantation until the time of transplantation, followed by two weekly maintenance doses of about 20 mg/kg of said anti-C5 antibody.

In one embodiment, tesidolumab or an antigen binding fragment thereof, preferably tesidolumab, is administered to a transplant candidate during said maintenance period at a dose of about 20 mg/kg at least weekly, at least bi-weekly, at least monthly over the period of at least 6 weeks, at least 9 weeks, at least 3 months, at least 6 months, at least 9 months, at least one year, lifelong. In one embodiment, tesidolumab or an antigen binding fragment thereof, preferably tesidolumab, is administered to a patient during said maintenance period as a every two weeks administration of about 20 mg/kg of said antibody, preferably tesidolumab. The maintenance period lasts for at least 6 weeks, e.g. 3 months, e.g. 6 months, e.g. 9 months, e.g. one year, e.g. lifelong.

In an embodiment, tesidolumab or an antigen binding fragment thereof, is administered to a patient, e.g. a transplant candidate, as at least one (e.g. one) induction dose of 40 mg/kg within a time period from up to six hours prior to transplantation until the time of transplantation, followed by two weekly maintenance doses of 20 mg/kg, followed by a every two weeks administration of 20 mg/kg of said antibody for a period of at least 6 weeks, e.g. 3 months, e.g. 6 months, e.g. 9 months, e.g. one year, e.g. lifelong.

The term “administering” encompasses administration of tesidolumab or an antigen binding fragment of the present invention, e.g. tesidolumab, in a single or multiple intravenous or subcutaneous doses.

In one embodiment, tesidolumab or an antigen binding fragment of the present invention is administered intravenously. For example, the induction dose and/or the maintenance dose is administered intravenously.

In a specific embodiment, tesidolumab or an antigen binding fragment thereof, is administered intravenously to a patient, e.g. a transplant candidate, as at least one (e.g. one) infusion dose of about 40 mg/kg at the time of transplantation followed by two weekly maintenance doses of 20 mg/kg, followed by a every two weeks administration of 20 mg/kg of said antibody, preferably tesidolumab for a period of at least 6 weeks, e.g. 3 months, e.g. 6 months, e.g. 9 months, e.g. one year, e.g. lifelong.

In another embodiment, tesidolumab or an antigen binding fragment of the present invention is administered subcutaneously. The “induction phase” and “maintenance period” doses should be adjusted for subcutaneous administration.

In pre-sensitized patients, antibody removal therapies such as plasma exchange (PE) or high dose IVIG can be used prior to and during the first 2-4 weeks post-transplant. The most common type of PE is plasmapheresis (PP), with albumin being the most common replacement fluid used. It can be performed on alternate days with a 1-1.5 fold-volume exchange with albumin or fresh frozen plasma. After multiple sessions circulating immunoglobulin concentrations can be effectively reduced through dilution and indiscriminate removal of all immunoglobulins. Immunoadsorption (IA) is another common type of antibody reduction therapy that can be used (e.g. outside of the US); it is more specific and more effective in reducing circulating immunoglobulins without the need for plasma substitution. IA is efficient in removing only IgG antibodies and capable of removing >85% of all circulating IgG during one session (Schwenger & Morath (2010), Nephrol Dial Transplant. 25(8): 2407-13). While this high specificity for IgG is useful for pathogenic IgG antibodies the lack of discrimination between endogenous and therapeutic IgG mAbs will result in the need for replacement of therapeutic monoclonal antibodies removed by this therapy as well as PP. Thus, in one embodiment, a supplemental dose of tesidolumab or an antigen binding fragment thereof, of at least 10 mg/kg, e.g. at least 20 mg/kg, e.g. eat least 30 mg/kg, e.g. at least 40 mg/kg, preferably 10 mg/kg, more preferably 20 mg/kg, is administered. For example such a supplemental dose is administered following completion of each PP or IA session, e.g. within 120 minutes following completion of each PP or IA session. For example, at least one supplemental dose is administered during the first 2-4 weeks post-transplant.

According to the present invention, tesidolumab may be administered to a patient who is a treatment-naïve patient, e.g. was not previously subjected to any anti-C5 antibody (such as tesidolumab or eculizumab) or antigen fragment thereof treatment. The population of anti-C5 antibody-naïve patients encompasses two different groups: (a) newly diagnosed cases; and (b) diagnosed patients who do not have access to anti-C5 antibodies.

In another embodiment, tesidolumab is administered to a patient who was previously subjected to treatment with an anti-C5 antibody or antigen fragment thereof, in particular eculizumab treatment. In another embodiment, a patient has been treated with an anti-C5 antibody different from tesidolumab or an antigen binding fragment thereof, in particular eculizumab, and wherein the patient is not responsive to said previous treatment.

In accordance with the present invention, tesidolumab or an antigen binding fragment thereof maybe administered to a patient in a pharmaceutical composition. In certain embodiments, tesidolumab or an antigen binding fragment thereof is a sole/single agent administered to the patient.

In another embodiment, tesidolumab or an antigen binding fragment thereof, is administered in combination with one or more other therapies, e.g. selected from the group consisting of cyclosporine, tacrolimus, mycophenolate mofetil,(MMF), myfortic, basiliximab, methotrexate, and corticosteroids, e.g. in addition to a triple therapy of e.g. cyclosporine (or tacrolimus) and mycophenolate mofetil (MMF) (or myfortic) and corticosteroids.

In particular, the following immunosuppressive treatment can be given in combination with the method according to the invention:
transplant induction therapy, such as:
   Anti-thymocyte globulin (rATG; e.g. Thymoglobulin), such as 15 mg lyophilized vial for IV administration following reconstitution with sterile water for injection;
   Basiliximab (e.g. Simulect®), e.g. as 20 mg lyophilized vial for IV administration following reconstitution with sterile water for injection.
transplant immunosuppressive maintenance therapy, such as:
   Tacrolimus, optionally combined with mycophenolate and/or corticosteroids, e.g. administered locally and dosed per local treatment protocol in accordance with local labeling. Baseline immunosuppression may be used according to the label;
   Tacrolimus (e.g. Prograf®) as 0.5 mg, 1.0 mg or 5.0 mg capsules or tablets or 5 mg/mL for injection;
   Mycophenolate mofetil (e.g. MMF, CellCept®) 250 mg or 500 mg film-coated tablets, or 250 mg capsules, or 500 mg vial for IV administration or enteric coated mycophenolate sodium (e.g. ECMPS; Myfortic®) as 180 or 360 mg tablets;
   Cyclosporine
   Methotrexate

In another embodiment, tesidolumab or an antigen binding fragment thereof, is administered without any immunosuppressive therapy or drug, e.g. without transplant induction therapy and/or without transplant immunosuppressive maintenance therapy. For example, tesidolumab or an antigen binding fragment thereof, is administered without administering tacrolimus (or cyclosporine), mycophenolate nor corticosteroids.

The following examples illustrate the invention described above, but are not, however, intended to limit the scope of the invention in any way. Other test models known as such to the person skilled in the pertinent art can also determine the beneficial effects of the claimed invention.

TABLE 1
Sequences

| SEQ | | |
|---|---|---|
| ID NO. | Information | Sequence |
| 1 | tesidolumab | SYAIS |
| | HCDR1 | |
| 2 | tesidolumab | GIGPFFGTANYAQKFQG |
| | HCDR2 | |
| 3 | tesidolumab | DTPYFDY |
| | HCDR3 | |
| 4 | tesidolumab | SGDSIPNYYVY |
| | LCDR1 | |
| 5 | tesidolumab | DDSNRPS |
| | LCDR2 | |
| 6 | tesidolumab | QSFDSSLNAEV |
| | LCDR3 | |
| 7 | tesidolumab VH | EVQLVQSGAEVKKPGSSVKVSCKASGGTFSSYAISW |
| | | VRQAPGQGLEWMGGIGPFFGTANYAQKFQGRVTITA |
| | | DESTSTAYMELSSLRSEDTAVYYCARDTPYFDYWGQ |
| | | GTLVTVSS |
| 8 | tesidolumab VL | SYELTQPLSVSVALGQTARITCSGDSIPNYYVYWYQQ |
| | | KPGQAPVLVIYDDSNRPSGIPERFSGSNSGNTATLTIS |
| | | RAQAGDEADYYCQSFDSSLNAEVFGGGTKLTVL |
| 9 | tesidolumab HC | EVQLVQSGAEVKKPGSSVKVSCKASGGTFSSYAISW |
| | | VRQAPGQGLEWMGGIGPFFGTANYAQKFQGRVTITA |
| | | DESTSTAYMELSSLRSEDTAVYYCARDTPYFDYWGQ |
| | | GTLVTVSSASTKGPSVFPLAPSSKSTSGGTAALGCLV |
| | | KDYFPEPVTVSWNSGALTSGVHTFPAVLQSSGLYSLS |
| | | SVVTVPSSSLGTQTYICNVNHKPSNTKVDKRVEPKSC |
| | | DKTHTCPPCPAPEAAGGPSVFLFPPKPKDTLMISRTP |
| | | EVTCVVVDVSHEDPEVKFNWYVDGVEVHNAKTKPRE |
| | | EQYNSTYRVVSVLTVLHQDWLNGKEYKCKVSNKALP |
| | | APIEKTISKAKGQPREPQVYTLPPSREEMTKNQVSLTC |
| | | LVKGFYPSDIAVEWESNGQPENNYKTTPPVLDSDGSF |
| | | FLYSKLTVDKSRWQQGNVFSCSVMHEALHNHYTQKS |
| | | LSLSPGK |
| 10 | tesidolumab LC | SYELTQPLSVSVALGQTARITCSGDSIPNYYVYWYQQ |
| | | KPGQAPVLVIYDDSNRPSGIPERFSGSNSGNTATLTIS |
| | | RAQAGDEADYYCQSFDSSLNAEVFGGGTKLTVLGQP |
| | | KAAPSVTLFPPSSEELQANKATLVCLISDFYPGAVTVA |
| | | WKADSSPVKAGVETTTPSKQSNNKYAASSYLSLTPEQ |
| | | WKSHRSYSCQVTHEGSTVEKTVAPTECS |

### EXAMPLES

### Example 1

The relationship between serum concentrations of total tesidolumab and serum complement activity was determined. An analysis of these data showed that concentrations of total tesidolumab that were below 55 μg/mL resulted in less than full suppression of serum complement activity.

By use of modelling, the relationship between tesidolumab dose and exposure indicates that doses of 20 mg/kg every two weeks are adequate to ensure inhibition of complement activity. According to the model, less than 0.5% of the patients would have exposure values at a trough level below the 55 μg/ml limit.

Based on the relationship between total serum concentrations of tesidolumab and serum complement activity, it has now been discovered that concentrations of total serum tesidolumab that were <55 μg/mL resulted in less than full suppression of serum complement activity. Therefore, a minimum total serum concentration of tesidolumab of 55-100 μg/mL is adequate to ensure inhibition of complement activity.

### Example 2

For a Phase 2 study on the prevention of antibody-mediated rejection (AMR) after kidney transplantation, two cohorts of pre-sensitized kidney transplant recipients (KTR) who are at high- or moderate-risk of developing AMR will be recruited. 48 KTR will be enrolled based on their immunologic risk as defined by pre-existing donor specific antibody concentrations (DSA) and a functional assessment of immunologic risk based on B-cell flow cytometry cross matching (BFXM). Both cohorts are to receive the same treatment regimen with tesidolumab in addition to their conventional immunosuppressive therapy and local pre- and post-transplant desensitization.

Tesidolumab is to be administered via intravenous (IV) infusion at the time of transplantation, prior to allograft revascularization and unclamping, using a body weight adjusted dose of 40 mg/kg tesidolumab. This initial dose is to be followed by two (2) weekly doses of 20 mg/kg tesidolumab IV and subsequently by a maintenance period using 20 mg/kg IV tesidolumab every 2 weeks thereafter. The core treatment period will last 12 months and will be followed by a 24 months tesidolumab treatment-free follow-up period for a total study duration of up to 36 months. The efficacy of tesidolumab in this Phase 2 trial will be measured by the incidence of acute and chronic AMR at 12 month post-transplantation.

### Populations

Pre-sensitized kidney transplant candidates are to be selected on the basis of pre-transplant DSA at the time of transplant as measured by a commercially available Luminex-based solid phase multiplex-bead assay (SAB) and B-cell flow cytometry cross-matching (BFXM) as measured by the local HLA laboratory.

High-risk candidates are defined as those who are CDC-crossmatch negative with a SAB MFI (as determined on the day of transplantation) greater than 5000 and BFXM greater than 250 whereas moderate-risk candidates will be defined as those who are CDC-crossmatch negative with a SAB MFI (as determined on the day of transplantation) from 3000 to 5000 and a BFXM less than 250.

### Dosing Regimen

An induction dose of 40 mg/kg tesidolumab will be administered prior to revascularization to ensure complete C5 blockade prior to exposing the allograft to the recipient's pre-formed anti-HLA antibodies. This induction dose with 40 mg/kg IV at the time of transplant will then be followed by two (2) weekly doses of 20 mg/kg tesidolumab to bind any remaining donor C5 in the allograft as well as suppress recipient C5 in the serum. Thereafter, a maintenance regimen using 20 mg/kg IV tesidolumab every 2 weeks, to bind newly synthesized recipient C5 and inhibit terminal complement activation, is planned for all KTR enrolled. Furthermore, supplemental administration of tesidolumab may be required after plasma exchange therapies and/or IVIG in order to replace tesidolumab removed from the vascular compartment by means of these therapeutic procedures. The supplemental administration is to be 20 mg/kg in the first three weeks. Afterwards, the supplemental administration is to be 10 mg/kg.

### Duration of Treatment

The Phase 2 trial includes a 12 month core treatment period and a 24 month follow-up period for a total study duration of up to 36 months.

### Primary and Secondary Endpoints

The same primary and secondary endpoints are to be assessed in both the high- and moderate-risk KTR. The primary end points include the effect of tesidolumab on safety, tolerability and incidence rate of AMR at month 12 post-transplant. Secondary endpoints include the incidence of transplant glomerulopathy (TG), as well as the incidence of scAMR and composite efficacy failure endpoints defined as: AMR, graft loss or death with/without loss-to follow-up as well as TG, graft loss or death with/without loss-to follow-up at month 12 post transplant.

1. Tesidolumab or an antigen binding fragment thereof for use in the prevention of transplant rejection e.g. in pre-sensitized patients.

2. Tesidolumab or an antigen binding fragment thereof for use in the prevention or treatment of AMR, e.g. acute AMR, e.g. subclinical AMR, e.g. chronic AMR, or a condition associated thereof.

3. Tesidolumab or an antigen binding fragment thereof for use according to claim 2 in the prevention or treatment of transplant glomerulopathy (TG).

4. Tesidolumab or an antigen binding fragment for use according to any of the preceding claims, wherein the patient is characterized by MFI equal to or greater than 5000 or comprised between 2000 and 10000.

5. Tesidolumab or an antigen binding fragment for use according to any of the preceding claims, wherein the patient is characterized by BFXM equal to or greater than 250 or comprised between 150 and 500.

6. Tesidolumab or an antigen binding fragment for use according to anyone of claims 1 to 3, wherein the patient is characterized by i) either MFI comprised between 2000 and 10000 and BFXM comprised between 150 and 500; or ii) MFI equal to or greater than 5000 and/or BFXM equal to or greater than 250.

7. Tesidolumab or an antigen binding fragment for use according to anyone of claims 1 to 3, wherein the patient is characterized by BFXM equal to or less than 250 or comprised between 150 and 250.

8. Tesidolumab or an antigen binding fragment for use according to anyone of claims 1 to 3, wherein the patient is characterized by MFI comprised between 3000 and 5000 and BFXM less than 250.

9. Tesidolumab or an antigen binding fragment for use according to any of the preceding claims, wherein the patient is CDC-crossmatch negative.

10. Tesidolumab or an antigen binding fragment thereof for use according to any of the preceding claims, wherein tesidolumab or the antigen binding fragment thereof is administered at least once at a dose of at least 20 mg/kg.

11. Tesidolumab or an antigen binding fragment thereof for use according to any of the preceding claims, wherein tesidolumab or the antigen binding fragment thereof is administered weekly or every two weeks.

12. Tesidolumab or an antigen binding fragment thereof for use according to any of the preceding claims, wherein tesidolumab or the antigen binding fragment thereof is administered for a period of at least 1 month, or at least 3 months, or at least 6 months, or at least one year, or lifelong.

13. Tesidolumab or an antigen binding fragment thereof for use according to any of the preceding claims, wherein tesidolumab or the antigen binding fragment thereof is administered repeatedly at a dose of at least 20 mg/kg and wherein the interval between two administrations is less than one month.

14. Tesidolumab or an antigen binding fragment thereof for use according to any of the preceding claims, wherein tesidolumab or the antigen binding fragment thereof is administered at a dose of at least 20 mg/kg weekly for a period of at least 2 weeks to 6 months, and is then administered at a dose of at least 20 mg/kg every two weeks for at least 3 months.

15. Tesidolumab or an antigen binding fragment thereof for use according to any of the preceding claims, wherein tesidolumab or the antigen binding fragment thereof is administered as an induction dose of at least about 40 mg/kg.

16. Tesidolumab or an antigen binding fragment thereof for use according to claim 15, wherein the induction dose is administered prior to transplantation or on the day of transplantation.

17. Tesidolumab or an antigen binding fragment thereof for use in the prevention or treatment of AMR or an associated condition thereof (e.g. TG) in a patient, wherein tesidolumab or said antigen binding fragment thereof is administered such that a constant serum trough level at steady-state of antibody (e.g. total antibody) of 10-100 μg/mL is maintained.

18. Tesidolumab or an antigen binding fragment thereof for use according to any of the preceding claims, wherein at least one supplemental dose of at least 10 mg/kg is administered to the patient, e.g. during the first 2 to 4 weeks after transplantation.

19. Tesidolumab or an antigen binding fragment thereof for use according to any of the preceding claims, wherein the patient is a solid organ transplant patient, e.g. a kidney transplant patient.

20. Tesidolumab or an antigen binding fragment thereof for use in a method of prevention transplant rejection or for the prevention or treatment of AMR (e.g. prevention of AMR) or an associated condition thereof in a transplant patient, wherein the method comprises the steps of:
a. identifying a patient having (as defined prior to transplantation) either i) MFI comprised between 3000 and 5000 and optionally BFXM equal to or less than 250, or ii) MFI equal to or greater than 5000 and/or BFXM equal to or greater than 250; and
b. administering tesidolumab or an antigen binding fragment thereof to the patient identified in step a) continuously for at least 3 months at a dose of at least 20 mg/kg at least every two weeks (or such that a constant plasma trough level at steady-state of total antibody of 10-100 μg/mL is maintained).

21. Use of tesidolumab or an antigen binding fragment thereof for the manufacture of a medicament (a) for prevention of transplant rejection e.g. in pre-sensitized patients, or (b) for the prevention or treatment of AMR, e.g. acute AMR, e.g. chronic AMR, or a condition associated thereof, e.g. transplant glomerulopathy (TG).

22. A method of preventing transplant rejection in a patient in need thereof, e.g. in a pre-sensitized patient, or to prolong graft survival in a patient in need thereof, e.g. in a pre-sensitized patient comprising administering to said patient a therapeutically effective amount of tesidolumab or an antigen binding fragment thereof.

23. A method of preventing or treating, AMR, e.g. acute AMR, e.g. chronic AMR, or a condition associated thereof, e.g. transplant glomerulopathy (TG), comprising administering to a patient a therapeutically effective amount of tesidolumab or an antigen binding fragment thereof.
